(19) United States
(12) Patent Application Publication
(10) Pub. No.: US 2025/0136913 A1
(43) Pub. Date: May 1, 2025

(54) IMPELLER AND BIOREACTOR INCLUDING THE SAME

(71) Applicant: Pensees Inc., Seoul (KR)

(72) Inventors: Chang Ve CHEAH, Seoul (KR); Byeongseon KANG, Seoul (KR)

(73) Assignee: Pensees Inc., Seoul (KR)

(21) Appl. No.: 18/506,323

(22) Filed: Nov. 10, 2023

(30) Foreign Application Priority Data
Oct. 27, 2023 (KR) 10-2023-0145518

(51) Int. Cl. C12M 1/06

(52) U.S. Cl. CPC C12M 27/02

(57) ABSTRACT

Provided are an impeller and a bioreactor including the same in which the impeller includes an extension that extends in up and down directions, a seating part coupled with the extension such that the extension passes through the center of the seating part, a blade that is coupled to a portion of a periphery of the seating part and that protrudes in a peripheral outward direction of the seating part, and a cover that covers the rest other than the portion of the periphery of the seating part in the peripheral outward direction.

### TECHNICAL FIELD

The present disclosure relates to an impeller and a bioreactor including the same.

### BACKGROUND

Bioreactors refer to mechanical apparatuses designed and manufactured to create a biologically active environment. The bioreactors are containers that cause a chemical reaction of organic matter extracted from living things or biochemically active substances. This process may be aerobic or anaerobic. Based on an operating method, the bioreactors are divided into a batch bioreactor, a fed-batch bioreactor, and a continuous bioreactor (e.g., a continuous stirred reactor). An example of a continuous fermentor is a chemostat.

A bioreactor may be used to culture a piece of meat. The piece of meat refers to a cultured mass obtained by combining cell fragments, in which cells and supports are coupled, into a certain size and shape that gives the texture of meat. When a culture medium and the piece of meat are fed into the bioreactor, the bioreactor may induce cell growth through material exchange by stirring the culture medium containing the piece of meat.

An impeller method may be used for the bioreactor for culturing the piece of meat. The impeller method is a method in which an impeller that has blades and that is placed inside a reaction tank rotates to generate a flow of fluid inside the reaction tank.

However, although an impeller in a general form helps with the function of stirring the culture medium inside the bioreactor, the impeller may cause direct physical damage to the piece of meat. Accordingly, a method for minimizing the physical damage to the piece of meat while securing the stirring performance inside the bioreactor necessary for culturing the piece of meat is required.

To this end, a method of placing the piece of meat in a cage inside the bioreactor may be considered. However, since the shape and size of a typical impeller depends on the size and ratio of the interior of the bioreactor, the space in which the cage is placed may be limited depending on the size and location of the impeller, and using the impeller to maintain a homogeneous flow of the culture medium may cause excessive stress on the piece of meat located inside.

Furthermore, when the impeller and the cage are separated from each other, the setting time required for installation and disassembly is increased, and the number of working processes is increased. Therefore, there is a high possibility that the piece of meat is contaminated due to the increase in the time during which the piece of meat is exposed to the outside.

### SUMMARY

The present disclosure has been made to solve the above-mentioned problems occurring in the prior art while advantages achieved by the prior art are maintained intact.

An aspect of the present disclosure provides an impeller for efficiently using an inner space of a bioreactor while securing stirring performance required for culturing a piece of meat and a bioreactor including the same.

Another aspect of the present disclosure provides an impeller for minimizing physical damage to a piece of meat and a bioreactor including the same.

Another aspect of the present disclosure provides an impeller for reducing a possibility of cell destruction and contamination of a piece of meat with integrated optimal design capable of simplifying a working process and a bioreactor including the same.

The technical problems to be solved by the present disclosure are not limited to the aforementioned problems, and any other technical problems not mentioned herein will be clearly understood from the following description by those skilled in the art to which the present disclosure pertains.

According to an aspect of the present disclosure, an impeller includes an extension that extends in up and down directions, a seating part coupled with the extension such that the extension passes through the center of the seating part, a blade that is coupled to a portion of a periphery of the seating part and that protrudes in a peripheral outward direction of the seating part, and a cover that covers the rest other than the portion of the periphery of the seating part in the peripheral outward direction.

The seating part may include a plurality of seating members, and the plurality of seating members may be arranged in the up and down directions to have a separation space therebetween.

The blade may be coupled to portions of peripheries of the plurality of seating members, and among edges of the blade, an edge coupled with the portions of the peripheries of the seating members may have a helical shape extending in the up and down directions and having a diameter corresponding to diameters of the seating members.

The blade may be coupled to portions of peripheries of the plurality of seating members, and a lateral edge of the cover may have a shape corresponding to a shape of an edge coupled with portions of peripheries of the plurality of seating members among edges of the blade.

The cover may cover the plurality of seating members and the separation space in the peripheral outward direction.

The seating members may include an annular seating portion perpendicular to the extension and an upwardly protruding portion that protrudes upward from a radial outer end portion of the seating portion.

The blade may include a connecting region that connects one seating member and another seating member located above the one seating member.

A plurality of seating portion holes may be formed through the seating portion in the up and down directions.

The impeller may further include an upper plate part coupled with the extension such that the extension passes through the center of the upper plate part, the upper plate part being located over the uppermost seating member among the plurality of seating members. An upper end portion of the blade may be coupled to the upper plate part.

The upper plate part may include an annular upper plate portion perpendicular to the extension and located over the seating part and a downwardly protruding portion that protrudes downward from a radial outer end portion of the upper plate portion.

A plurality of upper plate holes may be formed through the upper plate portion in the up and down directions.

The cover may be removably coupled to a portion of a periphery of the upper plate portion and a portion of a periphery of the lowermost seating member among the plurality of seating members.

A plurality of cover holes may be formed through the cover in the peripheral outward direction.

The cover may include a contact region brought into contact with the seating part and a non-contact region not being brought into contact with the seating part, and the cover holes may be formed in the non-contact region.

The blade may include a plurality of blades spaced apart from each other in a circumferential direction of the seating part, and the cover may include as many covers as the number corresponding to the number of blades.

The extension may be formed in a tubular shape having an inner passage and extending in the up and down directions.

An edge of the blade in the peripheral outward direction may be formed in a helical shape extending in the up and down directions and having a larger diameter than the seating part.

According to another aspect of the present disclosure, a bioreactor includes a tank in which a culture medium is accommodated and an impeller that is disposed in the tank and that stirs the culture medium and accommodates a piece of meat. The impeller includes an extension that extends in up and down directions, a seating part coupled with the extension such that the extension passes through the center of the seat part, the piece of meat being seated on the seating part, and a blade that is coupled to a portion of a periphery of the seating part and that protrudes in a peripheral outward direction of the seating part.

### BRIEF DESCRIPTION OF THE DRAWINGS

The above and other objects, features and advantages of the present disclosure will be more apparent from the following detailed description taken in conjunction with the accompanying drawings:

FIG. 1 is a sectional view of a bioreactor including an impeller according to an embodiment of the present disclosure;

FIG. 2 is a perspective view of the impeller according to an embodiment of the present disclosure;

FIG. 3 is a sectional view of the impeller according to an embodiment of the present disclosure;

FIG. 4 is a view illustrating a state in which a cover is removed in FIG. 2;

FIG. 5 is a view illustrating the cover; and

FIGS. 6 to 8 are views successively illustrating a method of placing a piece of meat in the impeller according to an embodiment of the present disclosure.

### DETAILED DESCRIPTION

Hereinafter, some embodiments of the present disclosure will be described in detail with reference to the exemplary drawings. In adding the reference numerals to the components of each drawing, it should be noted that the identical or equivalent component is designated by the identical numeral even when they are displayed on other drawings. Further, in describing the embodiment of the present disclosure, a detailed description of well-known features or functions will be ruled out in order not to unnecessarily obscure the gist of the present disclosure.

In this specification, front and rear directions, left and right directions, up and down directions, and vertical directions are referred to for convenience of description and may be perpendicular to one another. However, these directions may be determined relative to the direction in which an impeller 2 is arranged, and the up and down directions may not necessarily mean the vertical directions.

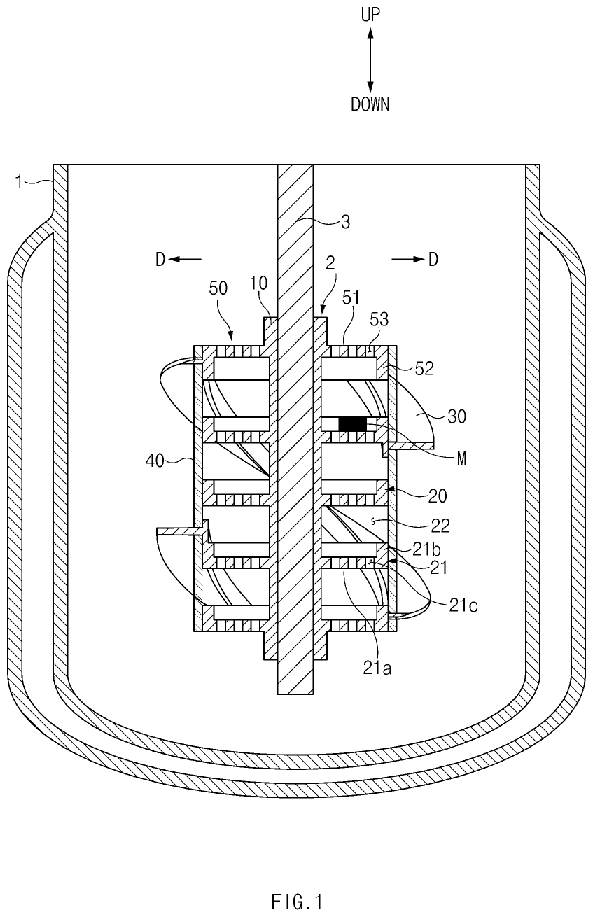

The impeller 2 according to an embodiment of the present disclosure relates to an impeller that is able to be used in a bioreactor for culturing a piece of meat M. FIG. 1 is a sectional view of the bioreactor including the impeller according to an embodiment of the present disclosure.

As illustrated in FIG. 1, the bioreactor may include a tank 1 and the impeller 2. A culture medium may be accommodated in the tank 1. The impeller 2 may be disposed in the tank 1 and may stir the culture medium. Furthermore, the piece of meat M may be accommodated in the impeller 2.

Hereinafter, the form of the impeller 2 will be described in detail.

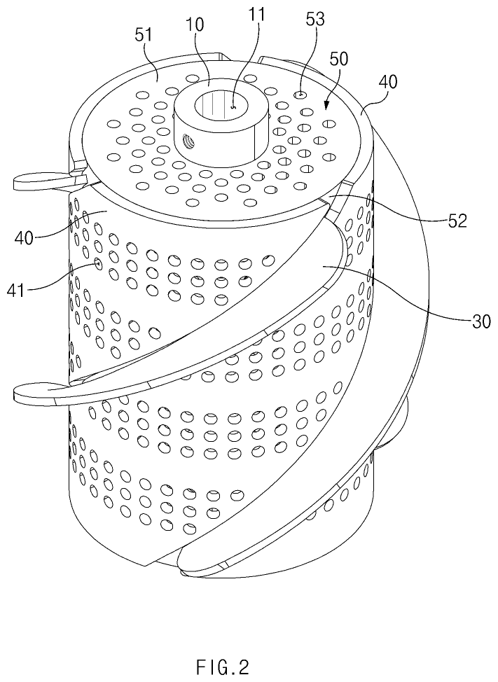

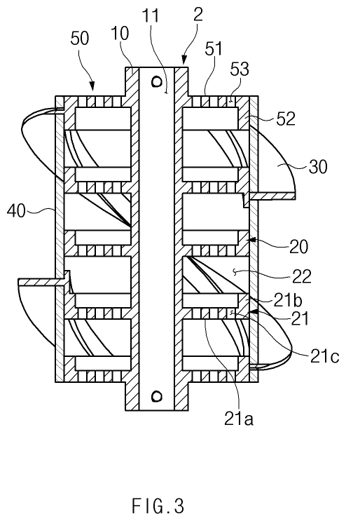

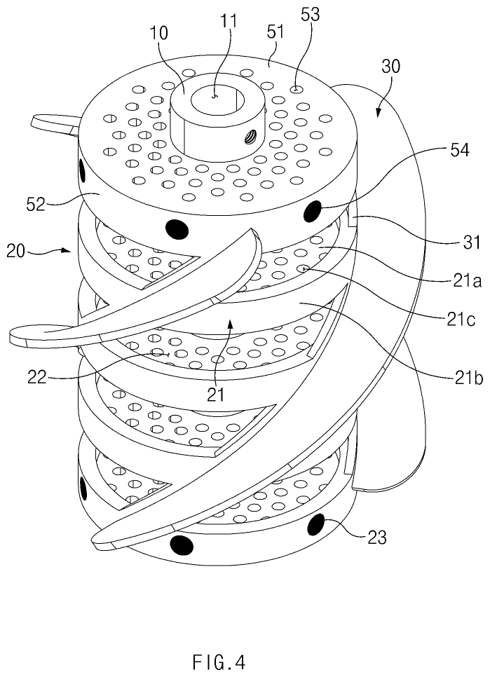

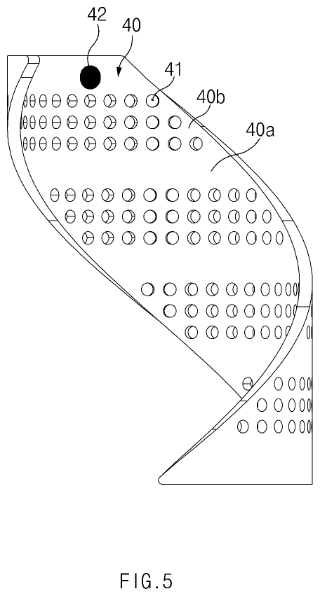

FIG. 2 is a perspective view of the impeller according to an embodiment of the present disclosure. FIG. 3 is a sectional view of the impeller according to an embodiment of the present disclosure. FIG. 4 is a view illustrating a state in which a cover is removed in FIG. 2. FIG. 5 is a view illustrating the cover.

The impeller 2 according to an embodiment of the present disclosure may include an extension 10, a seating part 20, a blade 30, and the cover 40. The extension 10 may extend in the up and down directions. The extension 10 may be a part connected with a shaft 3 that is disposed in the tank 1 so as to be rotatable. For example, the extension 10 may be formed in a tubular shape having an inner passage 11 and extending in the up and down directions. The shaft 3 may be coupled to the inner passage 11. As the shaft 3 rotates, the impeller 2 may rotate and stir the culture medium in the tank 1.

The seating part 20 may be coupled with the extension 10 such that the extension 10 passes through the center of the seating part 20. The seating part 20 may be a component on which the piece of meat M is seated.

The blade 30 may be coupled to a portion of a periphery of the seating part 20 and may protrude in a peripheral outward direction D of the seating part 20. The blade 30 may be a part for stirring the culture medium.

In the peripheral outward direction D, the cover 40 may cover the rest other than the portion of the periphery of the seating part 20.

Since the seating part 20 on which the piece of meat M is seated and the blade 30 for stirring the culture medium are coupled with each other in the impeller 2 according to an embodiment of the present disclosure, it is possible to efficiently use the inner space of the bioreactor while securing stirring performance required for culturing the piece of meat M.

In addition, since the cover 40, which covers the seating part 20, and the blade 30 are located on the outside of the seating part 20 according to the present disclosure, it is possible to minimize stress applied to the piece of meat M.

### Seating Part 20

The seating part 20 may include a plurality of seating members 21. The plurality of seating members 21 may be arranged in the up and down directions so as to have a separation space 22 therebetween. The cover 40 may cover the seating members 21 and the separation spaces 22 in the peripheral outward direction D.

Each of the seating members 21 may include a seating portion 21a and an upwardly protruding portion 21b. The seating portion 21a may be an annular portion perpendicular to the extension 10. The seating portion 21a may be a portion on which the piece of meat M is seated.

The upwardly protruding portion 21b may be a portion protruding upward from a radially outer end portion of the seating portion 21a. The upwardly protruding portion 21b may be a portion that obstructs movement of the piece of meat M seated on the seating portion 21a in the radially outward direction of the seating portion 21a.

A plurality of seating portion holes 21c may be formed through the seating portion 21a in the up and down directions. Since the seating portion holes 21c are formed in the seating portion 21a, the culture medium may be brought into contact with a surface of the piece of meat M adjacent to the seating portion 21a. In addition, the culture medium may be smoothly moved through the seating portion holes 21c in the inner space of the impeller 2, and thus the piece of meat M may be effectively cultured.

### Blade 30

The blade 30 may be coupled to portions of peripheries of the plurality of seating members 21. In addition, the blade 30 may include a connecting region 31. The connecting region 31 may be a region that connects one seating member 21 and another seating member 21 located above the one seating member 21. This may mean that the blade 30 has a form that connects the plurality of seating members 21.

For example, the portions of the peripheries of the plurality of seating members 21 to which the blade 30 is coupled may be located in different positions when viewed in the up and down directions.

Among edges of the blade 30, an edge coupled with the portions of the peripheries of the seating members 21 may have a helical shape extending in the up and down directions and having a diameter corresponding to the diameters of the seating members 21. In addition, an edge of the blade 30 in the peripheral outward direction D may be formed in a helical shape extending in the up and down directions and having a diameter greater than the diameter of the seating part 20.

However, the shape of the blade 30 is not limited thereto, and various modifications and changes may be made to the shape of the blade 30 within the scope of common general knowledge of those skilled in the art. For example, the edge of the blade 30 in the peripheral outward direction D may be formed in a straight line, or the edge facing away from the edge of the blade 30 in the peripheral outward direction D may be formed in a straight line.

The blade 30 may include a plurality of blades 30 spaced apart from each other in the circumferential direction of the seating part 20. As the plurality of blades 30 are provided, the stirring performance of the culture medium may be further enhanced. Meanwhile, the cover 40 that will be described below may include as many covers as the number corresponding to the number of blades 30. This may be the number corresponding to the number of spaces divided by the blades 30.

### Cover 40

The cover 40 may cover the remaining regions other than the regions to which the blades 30 are coupled, among the regions defined by the seating part 20. The cover 40 may cover the seating members 21 and the separation spaces 22 in the peripheral outward direction D.

The cover 40 may be coupled to the seating part 20 so as to be removable. For example, the cover 40 may be separated from and coupled to the seating part 20 through magnets. To this end, an upper magnet member 54 may be coupled to an upper plate part 50 that will be described below, a lower magnet member 23 may be coupled to the lowermost seating member 21, and cover magnet members 42 may be coupled to upper and lower ends of the cover 40. However, without being limited thereto, various modifications and changes may be made within the scope of common general knowledge of those skilled in the art. For example, the cover 40 may be separated from and coupled to the seating part 20 through an engagement structure, such as snap fit, or an adhesive structure.

Since the impeller 2 according to an embodiment of the present disclosure includes the detachable cover 40, a working process may be simplified when the piece of meat M is disposed, and thus a possibility of contamination of the piece of meat M may be reduced. In addition, the cover 40 may prevent cells of the piece of meat M from being damaged by the blades 30.

A lateral edge of the cover 40 may have a shape corresponding to the shape of the edge coupled with the portions of the peripheries of the plurality of seating members 21 among the edges of the blade 30. That is, the lateral edge of the cover 40 may have a helical shape.

A plurality of cover holes 41 may be formed through the cover in the peripheral outward direction D. Since the cover 40 has the cover holes 41, the culture medium may be smoothly introduced into the impeller 2, and movement of the piece of meat M out of the impeller 2 may be obstructed so that the piece of meat M may be prevented from being directly damaged by the blade 30.

The cover 40 may include a contact region 40a and a non-contact region 40b. The contact region 40a may be a region brought into contact with the seating part 20. The non-contact region 40b may be a region that is not brought into contact with the seating part 20. The non-contact region 40b may be a region that covers the separation space 22. The cover holes 41 may be formed in the non-contact region 40b.

Since the contact region 40a is a region blocked by the seating part 20, the culture medium is not able to be introduced into the cover 40 even though the cover holes 41 are formed. Meanwhile, when many cover holes 41 are formed, the rigidity of the cover 40 may be weakened. Accordingly, the cover holes 41 may not be formed in the contact region 40a through which the culture medium is not able to be introduced, and thus the rigidity of the cover 40 may be maintained.

### Upper Plate Part 50

The impeller 2 according to an embodiment of the present disclosure may further include the upper plate part 50. The upper plate part 50 may be coupled with the extension 10 such that the extension 10 passes through the center of the upper plate part 50. The upper plate part 50 may be located over the uppermost seating member 21 among the plurality of seating members 21. The upper plate part 50 may serve to obstruct upward movement of the piece of meat M seated on the uppermost seating member 21 among the seating members 21.

An upper end portion of the blade 30 may be coupled to the upper plate part 50. The cover 40 may be removably coupled to a portion of a periphery of the upper plate part 50 and a portion of a periphery of the lowermost seating member 21 among the plurality of seating members 21.

The upper plate part 50 may include an upper plate portion 51 and a downwardly protruding portion 52. The upper plate portion 51 may be an annular portion that is perpendicular to the extension and located above the seating part 20. The downwardly protruding portion 52 may be a portion protruding downward from a radially outer end portion of the upper plate portion 51.

A plurality of upper plate holes 53 may be formed through the upper plate portion 51 in the up and down directions. The culture medium may be smoothly introduced below the upper plate part 50 through the upper plate holes 53.

### Method of Placing Piece of Meat

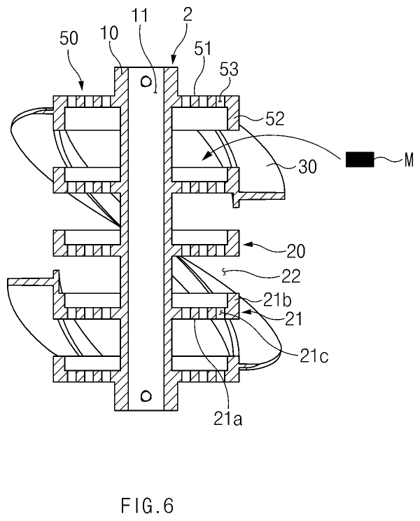

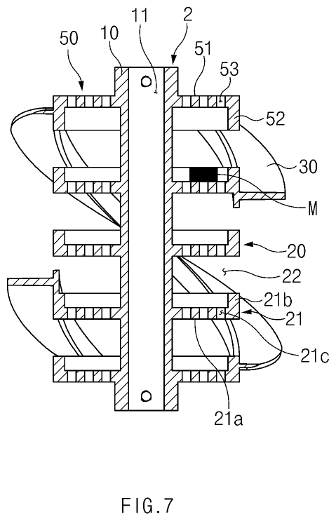

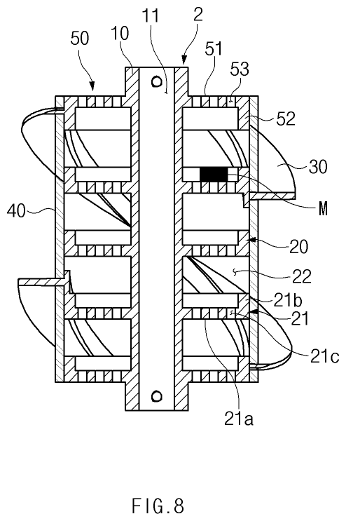

FIGS. 6 to 8 are views successively illustrating a method of placing a piece of meat in the impeller according to an embodiment of the present disclosure. Hereinafter, the method of placing the piece of meat M in the impeller 2 according to this embodiment will be described with reference to the above-described contents and FIGS. 6 to 8. The method of placing the piece of meat M in the impeller 2 may mean a process of seating the piece of meat M in the seating part of the impeller 2.

First, the cover 40, which are coupled to the seating part and the upper plate part 50, is separated.

Next, the piece of meat M is placed on the seating portion 21a of the seating part 20.

Finally, the cover 40 is coupled to the seating part 20 and the upper plate part 50, the shaft 3 and the extension 10 are coupled to each other, and thereafter the impeller 2 is placed in the tank 1. Accordingly, preparation for the culture of the piece of meat M is made.

According to the present disclosure, the cage structure and the blade structure are integrated with each other. Accordingly, it is possible to efficiently use the inner space of the bioreactor while securing the stirring performance required for culturing the piece of meat.

In addition, according to the present disclosure, the blade is located on the outside of the cage, and the detachable cover is disposed on the outside of the cage. Accordingly, it is possible to minimize stress applied to the piece of meat, simplify the working process, and reduce a possibility of cell destruction and contamination of the piece of meat.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims.

What is claimed is:

1. An impeller comprising:
an extension configured to extend in up and down directions;
a seating part coupled with the extension such that the extension passes through the center of the seating part;
a blade coupled to a portion of a periphery of the seating part and configured to protrude in a peripheral outward direction of the seating part; and
a cover configured to cover the rest other than the portion of the periphery of the seating part in the peripheral outward direction.

2. The impeller of claim 1, wherein the seating part includes a plurality of seating members, and
wherein the plurality of seating members are arranged in the up and down directions to have a separation space therebetween.

3. The impeller of claim 2, wherein the blade is coupled to portions of peripheries of the plurality of seating members, and
wherein among edges of the blade, an edge coupled with the portions of the peripheries of the seating members has a helical shape extending in the up and down directions and having a diameter corresponding to diameters of the seating members.

4. The impeller of claim 2, wherein the blade is coupled to portions of peripheries of the plurality of seating members, and
wherein a lateral edge of the cover has a shape corresponding to a shape of an edge coupled with portions of peripheries of the plurality of seating members among edges of the blade.

5. The impeller of claim 2, wherein the cover covers the plurality of seating members and the separation space in the peripheral outward direction.

6. The impeller of claim 2, wherein the seating members include:
an annular seating portion perpendicular to the extension; and
an upwardly protruding portion configured to protrude upward from a radial outer end portion of the seating portion.

7. The impeller of claim 6, wherein the blade includes a connecting region configured to connect one seating member and another seating member located above the one seating member.

8. The impeller of claim 6, wherein a plurality of seating portion holes are formed through the seating portion in the up and down directions.

9. The impeller of claim 2, further comprising:
an upper plate part coupled with the extension such that the extension passes through the center of the upper plate part, the upper plate part being located over the uppermost seating member among the plurality of seating members,
wherein an upper end portion of the blade is coupled to the upper plate part.

10. The impeller of claim 9, wherein the upper plate part includes:
an annular upper plate portion perpendicular to the extension and located over the seating part; and
a downwardly protruding portion configured to protrude downward from a radial outer end portion of the upper plate portion.

11. The impeller of claim 10, wherein a plurality of upper plate holes are formed through the upper plate portion in the up and down directions.

12. The impeller of claim 9, wherein the cover is removably coupled to a portion of a periphery of the upper plate portion and a portion of a periphery of the lowermost seating member among the plurality of seating members.

13. The impeller of claim 10, wherein a plurality of cover holes are formed through the cover in the peripheral outward direction.

14. The impeller of claim 13, wherein the cover includes:
a contact region brought into contact with the seating part; and
a non-contact region not being brought into contact with the seating part, and
wherein the cover holes are formed in the non-contact region.

15. The impeller of claim 1, wherein the blade includes a plurality of blades spaced apart from each other in a circumferential direction of the seating part, and
wherein the cover includes as many covers as the number corresponding to the number of blades.

16. The impeller of claim 1, wherein the extension is formed in a tubular shape having an inner passage and extending in the up and down directions.

17. The impeller of claim 1, wherein an edge of the blade in the peripheral outward direction is formed in a helical shape extending in the up and down directions and having a larger diameter than the seating part.

18. A bioreactor comprising:
a tank in which a culture medium is accommodated; and
an impeller disposed in the tank and configured to stir the culture medium, a piece of meat being accommodated in the impeller,
wherein the impeller includes:
an extension configured to extend in up and down directions;
a seating part coupled with the extension such that the extension passes through the center of the seat part, the piece of meat being seated on the seating part; and
a blade coupled to a portion of a periphery of the seating part and configured to protrude in a peripheral outward direction of the seating part.
